(19) United States
(12) Patent Application Publication
(10) Pub. No.: US 2023/0191023 A1
(43) Pub. Date: Jun. 22, 2023

(54) Wearable Fluid Delivery System Providing Regimen-Predictive Analytics

(71) Applicant: Luminoah, Inc., Charlottesville, VA (US)

(72) Inventors: Neal Piper, Charlottesville, VA (US); Hill Johnson, Charlottesville, VA (US); Brian J. Bergeron, Austin, TX (US)

(21) Appl. No.: 17/645,181

(22) Filed: Dec. 20, 2021

(51) Int. Cl. A61M 5/142; A61M 5/172; G16H 20/17

(52) U.S. Cl. CPC A61M 5/14248; A61M 5/1723; G16H 20/17; A61M 2005/1726; A61M 2005/14252; A61M 2205/3553

(57) ABSTRACT

The embodiments herein are directed to a portable liquid delivery system that incorporates an immersion driving pump mechanism(s), an administration reservoir, a user interface, and a wearable and/or portable accessory. The wearable accessory serves to house the components of the system and is to be worn on the user's body. In some cases, the system may be used to deliver nutritional formula for the treatment of patients on an enteral nutrition regimen. The apparatus described facilitates telehealth, training, and predictive processes to facilitate maximal therapeutic outcomes.

### COPYRIGHT NOTICE

A portion of the disclosure of this patent document contains material which is subject to copyright protection. The copyright owner has no objection to the facsimile reproduction of the patent document or the patent disclosure, as it appears in the Patent and Trademark Office patent file or records, but otherwise reserves all copyright rights whatsoever.

### BACKGROUND

Enteral nutrition, or tube feeding, is a process that delivers nutrition directly to the stomach or small intestine in place of traditional oral feeding. If a patient is receiving treatment outside of a hospital setting, the process is referred to as Home Enteral Nutrition (HEN). A 2013 study indicates that as many as 250,000 adults and 190,000 children currently require HEN as a part of their medical treatment in the United States. Currently, the leading conditions that indicate a need for HEN include cancer, nonmalignant respiratory disease, and neurological disorders. Enteral nutrition currently requires an array of medical resources and technologies including doctor assessment, a nutrition plan prescribed by a nutrition support team, a surgically implanted gastrostomy tube, a delivery system, tubing sets, and a nutritional formula.

Medical patients for whom oral feeding is not allowable or sufficient commonly benefit from prescribed enteral nutrition. This form of therapy delivers nutrition directly to a patient's gastrointestinal tract (GI) through man-made tubes that are placed into the GI tract. In order to access any portion of the patient's GI tract, the placed tubes must enter the patient's body through incisions created in the patient's abdominal wall or through existing body cavities such as the nasal cavity.

The distal end of any such tube is placed in the GI tract, while the proximal end of any such tube remains outside of the patient's body, permitting the proximal end to interface with enteral nutrition delivery technology. Surgically implanted tubes are generally indicated for long-term enteral nutrition needs while nasally placed tubes are indicated for short-term (less than two months) needs or when a patient is not healthy enough to tolerate surgery. Commonly, gastrostomy tubes are placed one of three ways: (1) surgically, through an open procedure or laparoscopically, (2) endoscopically, or (3) radiologically with a percutaneous insertion procedure.

### BRIEF DESCRIPTION OF THE DRAWINGS

Certain illustrative embodiments illustrating organization and method of operation, together with objects and advantages may be best understood by reference to the detailed description that follows taken in conjunction with the accompanying drawings in which:

FIG. 1 is a view of the device in-situ upon a patient's torso consistent with certain embodiments of the present invention.

FIG. 2A is a partially exploded front view of the device consistent with certain embodiments of the present invention.

FIG. 2B is a rear view of the device consistent with certain embodiments of the present invention.

FIG. 3A is a first detail view of the cross-cutaneous access portion of the device consistent with certain embodiments of the present invention.

FIG. 3B is a second detail view of the cross-cutaneous access portion of the device consistent with certain embodiments of the present invention.

FIG. 3C is a third detail view of the cross-cutaneous access portion of the device consistent with certain embodiments of the present invention.

FIG. 4 illustrates several smart-device-integrated user-experiences consistent with certain embodiments of the present invention.

FIG. 5 illustrates patient-remote diagnostic and therapeutic communications consistent with certain embodiments of the present invention.

### DETAILED DESCRIPTION

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language).

Reference throughout this document to “one embodiment”, “certain embodiments”, “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

Embodiments of the invention also include a computer readable medium comprising one or more computer files comprising a set of computer-executable instructions for performing one or more of the calculations, steps, processes and operations described and/or depicted herein. In exemplary embodiments, the files may be stored contiguously or non-contiguously on the computer-readable medium. Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” is a non-transitory computer-readable medium and includes any kind of computer memory such as floppy disks, conventional hard disks, CD-ROM, Flash ROM, non-volatile ROM, electrically erasable programmable read-only memory (EEPROM), and RAM. In exemplary embodiments, the computer readable medium has a set of instructions stored thereon which, when executed by a processor, cause the processor to perform tasks, based on data stored in the electronic database or memory described herein. The processor may implement this process through any of the procedures discussed in this disclosure or through any equivalent procedure.

In an embodiment of the invention, files comprising the set of computer-executable instructions may be stored in computer-readable memory on a single computer or distributed across multiple computers. A skilled artisan will further appreciate, in light of this disclosure, how the invention may be implemented, in addition to software, using hardware or firmware. As such, as used herein, the operations of the invention may be implemented in a system comprising a combination of software, hardware, or firmware.

Embodiments of this disclosure include one or more computers or devices loaded with a set of the computer-executable instructions described herein. The computers or devices may be a general purpose computer, a special-purpose computer, or other programmable data processing apparatus to produce a particular machine, such that the one or more computers or devices are instructed and configured to carry out the calculations, processes, steps, operations, algorithms, statistical methods, formulas, or computational routines of this disclosure. The computer or device performing the specified calculations, processes, steps, operations, algorithms, statistical methods, formulas, or computational routines of this disclosure may comprise at least one processing element such as a central processing unit (i.e., processor) and a form of computer-readable memory which may include random-access memory (RAM) or read-only memory (ROM). The computer-executable instructions may be embedded in computer hardware or stored in the computer-readable memory such that the computer or device may be directed to perform one or more of the calculations, steps, processes and operations depicted and/or described herein.

Additional embodiments of this disclosure comprise a computer system for carrying out the computer-implemented method of this disclosure. The computer system may comprise a processor for executing the computer-executable instructions, one or more electronic databases containing the data or information described herein, an input/output interface or user interface, and a set of instructions (e.g., software) for carrying out the method. The computer system may include a stand-alone computer, such as a desktop computer, a portable computer, such as a tablet, laptop, PDA, or smartphone, or a set of computers connected through a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth or other Near Field Communication (NFC) enabled network. In one embodiment, the computer system comprises a central computer connected to the internet that has the computer-executable instructions stored in memory that is operably connected to an internal electronic database. The central computer may perform the computer-implemented method based on input and commands received from remote computers through a network communications connection such as, but not limited to, the Internet. The central computer may effectively serve as a server and the remote computers may serve as client computers such that the server-client relationship is established, and the client computers issue queries or receive output from the server over a network.

The input/output interfaces may include a graphical user interface (GUI) which may be used in conjunction with the computer-executable code and electronic databases. The graphical user interface may allow a user to perform these tasks through the use of text fields, check boxes, pull-downs, command buttons, and the like. A skilled artisan will appreciate how such graphical features may be implemented for performing the tasks of this disclosure. The user interface may optionally be accessible through a computer connected to the internet. In one embodiment, the user interface is accessible by typing in an internet address through an industry standard web browser and logging into a web page. The user interface may then be operated through a remote computer (client computer) accessing the web page and transmitting queries or receiving output from a server through a network connection.

It is noted in particular that where a range of values is provided in this specification, each value between the upper and lower limits of that range is also specifically disclosed. The upper and lower limits of these smaller ranges may independently be included or excluded in the range as well.

Reference herein to “Disposable Pump Head” refers to a single use pump that is indicated for a set volume of use and then either discarded or, in a non-limiting embodiment, cleaned and repurposed for additional use in the system or for use in other systems.

Reference herein to “Pump Tubing” refers to all necessary tubing to connect a nutrient reservoir to a disposable pump head, and the disposable pump head to a patient access device. This may include a proprietary adaptor to connect directly to the patient's access device, such as, by way of non-limiting example, the Gastrostomy button (G-button) adapter developed for more secure connection for the patient access, but may simply be a standard connection to interface with an extension set that would then connect to patient's access device.

Reference herein to “Nutrient Reservoir” refers to a container that is used to hold enteral nutritional formula and deliver contents to the disposable pump head through the pump tubing. This container may come pre-filled and ready to directly insert into the proprietary “enLumin” system, or may come as a reusable and refillable container.

Reference herein to “Inlet Cxn” refers to the tubing that connects the nutrient reservoir to the disposable pump head.

Reference herein to “Outlet Cxn” refers to the tubing that connects the disposable pump head to the patient's access device or extension set.

The present invention is directed to an enteral nutrition system. Malnutrition and dysphagia are increasing, especially in chronic disease patients and elderly people. The occurrence of malnutrition is high in patients with chronic illnesses like cancer, neurological disorders, heart failure, and COPD, and increases with age as well. The prevalence of various cancers, especially gastric, head and neck/throat, and esophageal cancers, is growing globally, correlating to a rise in the need for enteral feeding in some oncology patients. Also, there is an increase in new markets where enteral feeding is playing a role for the first time. These include areas such as sports medicine and athletic training, pregnant women who suffer from hyperemesis gravidarum, and treatment for bulimia/anorexia conditions.

Despite the fact that many of the manufacturers of enteral feeding pumps claim that their equipment is designed to be portable, the current methods for HEN mobility are an afterthought in the form of inadequate, over-priced backpacks. Although backpacks appear to provide patients with increased mobility, treatments often fail when the pump is not positioned on an IV stand with a stationary patient. Mechanical failures of the device may also occur, and may include the occlusion of tubing as a result of kinking or viscous formula and/or the malalignment of the feeding bag in the backpack causing flow to be interrupted. As a result of these mobility issues during feeds, it is estimated that the average person is required to sit at least 3 hours per day to reach their required nutrition.

Accordingly, the need exists for an improved portable enteral nutrition system. In an embodiment, the innovative system herein described may comprise an in-line or immersion pump, an innovative administration reservoir, and a wearable housing that facilitates simplified portable feeding. In an embodiment, the innovative system may also be used when not attached to a wearable component of the enteral nutrition system, but may be utilized in connection to a non-portal system. In various embodiments, the improved enteral nutrition system may be optimized for portable use through connection to a wearable housing, or may be optimized for non-mobile use, or may be optimized for connection to existing, non-portable enteral nutrition feeding equipment.

In an embodiment, the instant innovation allows the patient full or near-full mobility as nutritive fluids are administered. In this way, a patient may find the product useful in their everyday life, as it may grant them autonomy by untethering them from a pole and machine that requires them to be immobile a good portion of the day. In addition, the system is a safer alternative than existing solutions because it takes away the risk of having tension applied to the extra slack of tubing, a situation which may cause problems with safety and efficacy.

The present invention is directed to an enteral nutrition system comprising an immersion fluid/nutrition driving mechanism, an administration reservoir, an electronic control and communication element, and a portable housing or accessory designed to contain the aforementioned components of the system. In an embodiment, the nutrition driving mechanism, in aspects, may include: a physical mechanism for sustained supply of liquid at a pump inlet; a device-specific attachment for securement to a wearable garment; a secure device-specific attachment to the user for perfusion or introduction of a liquid from a reservoir into the body of the user; direct or indirect connection to smart devices for communication with a controller and data collection; and associated sensors for detection of pump occlusions, priming completion, and liquid volume recording capabilities.

In an embodiment, the administration reservoir comprises a secure attachment to the wearable garment. In aspects, the enteral feeding device wraps around a patient's midsection and connects directly to a surgically implanted gastrostomy connection point. The connection point may consist of a gastronomy tube, a secure, grommet-like attachment point that permits ingress of a liquid into the body of the user, or other liquid introduction devices. A food pouch is capable of being inserted into the food reservoir section on the front of the wrap and a rolling mechanism pushes food to the feeding pump which then delivers the food directly into the patient's stomach or intestine through the connection point. The rate of feeding may be wirelessly controlled by a mobile application in communication with the electronic control and communication element. Sensors embedded within the wrap may also be capable of monitoring vitals like heart rate, feeding rate, user motion, or other vital parameters which may also be tracked/monitored via a mobile application.

In further embodiments, the present invention is directed at an immersion pumping system for the delivery of enteral nutrition formula to a patient through nasal, gastric, jejunal, or other intestinal access.

In an embodiment, the present invention is directed to an enteral nutrition device, wherein the fluid driver is capable of providing improved efficiencies because the pump may produce necessary flow rates for delivering nutrition in a smaller device footprint. The present invention may be comprised of a system with one or more pumping mechanisms to deliver fluid to a patient directly or from an attached reservoir that is integral to a wearable device. In a non-limiting example, the present invention may be directed to an enteral nutrition device, wherein the fluid driver is capable of providing improved portability because the system may operate with the administration reservoir horizontally arranged with respect to the pumping mechanism.

In an embodiment, the instant system may facilitate access and control by prescribing physicians and home health departments within large hospital systems. Prescribing physicians may be provided with access to synchronous or near-synchronous communication with the electronic communication element, providing insights into the home feeding environment and functioning of the system. These insights may include information regarding tracking features and processing utilizing one or more Machine Learning algorithms to assist with changes to the system and treatment. In a non-limiting example, doctors may have the ability to make informed decisions and identify inconsistencies between prescribed care and observed results when reviewing tracking data and analysis information from the enteral feeding system. From a home health perspective, the system may assist with and guide troubleshooting processes. To facilitate telehealth and home health use, the system may connect to one or more sensors and communicate operational data to an external display as a result of mobile device, smartphone or computer processor connectivity. The mobile device, smartphone, or computer processor connectivity grants remote access and control to the user, caregivers, and/or medical providers. Furthermore, the improved system and device may provide increased product life spans as a result of the portable design mitigating accidental damages due to user error or dropping hazards.

Regarding end-user patients, the system allows enhanced autonomy over patient feeds by having the ability to be ambulatory as they are using the device and system. The portable and ambulatory nature of the system may eliminate or nearly-eliminate a need for gravity-fed feeds, which allows the patient the freedom to receive a feeding without being tethered to a non-portable feeding mechanism, such as a physical stand. In addition, the wearable capability of the enteral feeding system may eliminate the need for extra tubing, which may be a tripping hazard and cause the tubing, fitted to the stomach in typical situations, to be ripped from the abdomen.

In an embodiment, the enteral feeding system may be relatively quiet, have long battery life, and be capable of low heat generation. The system may provide for the capability of connectivity with existing nasal tubes, G-tubes, J-tubes, and even GJ-tubes, or related technologies.

In an embodiment, the nutrition inlet device may provide a housing or container for a pouch capable of allowing insertion of nutrition products that are delivered at a controllable rate through the inlet via the pump device. The enteral feeding system may be integrated and communicate with a mobile device or smart phone application or other control application. This integration and communication capability permits the system to track fluid intake and feed times, provide notifications and other communications, and provides the ability to share data with caregivers, including a medical provider/physician/nurse/caregiver/family member. In an embodiment, the system may eliminate the difficult setup and manual priming actions currently required by patients, thus reducing the burden of effort required to initiate each feeding session. The device may also have a sufficiently low profile when being worn by a user that the system as a whole may be difficult to see, recognize, identify, or perceive by an outside observer.

In an embodiment, the enteral feeding system may be configured for prolonged delivery of nutritional formula during ambulation. The system may include one or more devices and components including, but not limited to, a housing containment garment configured to be worn on the body of the user, a pumping mechanism such as, in a non-limiting example, an immersion pump, inline pump, or other low volume/low profile pump, an electronic command and control device, and a liquid reservoir for introduction of the liquid into the body of a user through the pumping mechanism.

In an embodiment, a system is provided that may be configured for the delivery of a medical fluid, including a medication or other therapeutic fluid, for the treatment of a patient condition for a specified duration of time, for a particular time or treatment, or for a specific and controllable rate of delivery. In a non-limiting example, the system may be used by oncology patients that undergo continuous home infusion chemotherapy that share many of the treatment, safety, efficacy, and quality of life issues that enteral nutrition patients experience.

In an embodiment of the present invention, the system is capable of providing customized or standardized delivery of a nutritional formula configured in a system capable of being worn by the user. The system may include a garment worn on the body of the user, a pumping mechanism, such as a pump as previously described, a nutrient or fluid reservoir, and wireless or wired communication with an integrated computer or communication system/device (including, by way of non-limiting example, a mobile device, a smartphone, a computer, a computer processing user, and/or the cloud) which is capable of communicating with interested parties including but not necessarily limited to, the user, a relative of the user, caregiver, and/or a health care provider. In an embodiment, the device is capable of alerting the user or a health care provider with information related to the system, patient/user, treatment, device, fluid, or other aspects related to the system and system capabilities. In a non-limiting example, alarms or alerts may be transmitted to the mobile device, smartphone, computer, server, the cloud, or other remote electronic device. In a particular embodiment, the alarms or alerts may be delivered to a user via haptic feedback or may be visual, auditory, or textual in nature.

In an embodiment, the present innovation may be an enteral nutrition feeding system including an immersion or in-line pumping mechanism such as a pump as previously described, a fluid reservoir, a controller, and a wearable garment configured to contain the components of the system including but not limited to a pumping mechanism, the fluid reservoir, and control and communication electronics. In an embodiment, the pumping mechanism may include an apparatus capable of sustainably supplying a fluid at the pumping mechanism inlet, an attachment configured to secure the wearable garment to a user, a wireless or wired connection to an electronic device configured to communicate with the controller, the electronic control and communication apparatus, and one or more sensors for detecting one or more of pump occlusion, priming completion, liquid volume, and amount of liquid delivered. The control electronics may be active to collect and store all data related to fluid delivery, fluid volume, feeding schedule, and permit the information to be displayed and relayed to a user, caregiver, and/or medical professional.

In an embodiment, the instant innovation includes a fluid reservoir for holding enteral nutrition fluids or other fluids as required by user needs, that is worn in a belt or other wearable device external to a patient. The electronic command and control device for administering the enteral nutrition fluid via a pump is integrated into the belt or other wearable device. The instant innovation includes a drive motor operative to affect the action of a disposable pump for administration of the fluid. In an embodiment the drive motor is contained in the belt or other wearable device and as such does not become soiled by contact with the fluid passing from the fluid reservoir to the user. In an embodiment the disposable pump connects the reservoir of fluid to a G-button or similar cross-cutaneous mechanical connector.

In an embodiment the instant innovation may be used for Intermittent Infusions. This would include infusions up to 4 hours long for patients that may be able to tolerate higher flow rates during their feeds. These individuals would be able to charge their devices between use, if constrained by battery life.

In an embodiment the instant innovation may be used for Continuous Infusions. These may be appropriate for individuals that may be attached to a feeding machine for 12-24 hours per day. In a non-limiting example, such use may involve very low flow rates (below about 150 ml/hr) and supplies all hydration and nutrition needs for a single patient. These patients would require replaceable batteries or larger batteries to accommodate battery usage of up to 18 hours per day.

In an embodiment the instant innovation may be used for Night Feeds. These may include feedings for any user that requires infusions during their sleep to reach a certain level of nutrition. In a non-limiting example, these users may also use low flow rates (below about 150 ml/hr) and would ideally benefit from a battery life that would not require a wired connection for the overnight infusion.

In an embodiment the instant innovation may incorporate disposable elements into an integrated system. In an embodiment, a disposable pump head, pump tubing, and a pre-filled fluid reservoir may be replaced after each feeding. In an embodiment, the pre-filled fluid reservoir may be replaced after each feeding and the disposable pump head and pump tubing may be replaced after each day's feedings, or, optionally, after each use of the system. In an embodiment, each of the disposable pump head, pump tubing, and reusable fluid reservoir may be replaced after each day's feedings. In this embodiment, the fluid reservoir may be pre-filled with any fluid, such as, in non-limiting examples, nutrient fluids, medications, or other therapeutic fluids, that may be pumped from the fluid reservoir through the pump for delivery to the user.

In an embodiment of the system, a wearable garment may be configured to assist with a method of mobile fluid infusion, including, for example, the administration of therapeutic agents for medical purposes. In aspects, the system may use an immersion pumping mechanism placed against the abdomen to deliver fluid to a patient. The pumping mechanism may have components capable of removal for cleaning. The components may include a removable cartridge with built-in tubing, a removable top cap for troubleshooting, maintenance, and cleaning purposes, a removable portion of the pumping mechanism, and/or a disposable rotor for the pumping mechanism.

In an embodiment, the fluid reservoir may include one-way valve or two-way valves designed for refilling nutritional formula or other liquids into the fluid reservoir. In an embodiment, the fluid reservoir may include a specialized connection to the driving mechanism and/or it may include two specialized connections to two fluid driving mechanisms for delivery of the fluid from the fluid reservoir to the user. More specifically, the present system may include two driving mechanisms, such as a pump and a compressive rolling mechanism for the sustained delivery of nutrition, or the system may include two driving mechanisms, such as a pump and a pressurized compression system for the sustained delivery of fluids from the fluid reservoir.

In an embodiment, the portable housing may be configured to include a compression wrap garment that may be worn on the abdomen, back, or other body part of the user, wherein the housing would be capable of custom or standardized connections between the pumping mechanism(s) and the fluid reservoir. The housing may also be capable of facilitating connection of the system to the patient's surgical G-button connector, or other access site.

In a particular embodiment, the invention may include a portable nutrition delivery system that is less than 10 pounds, less than 9 pounds, less than 8 pounds, less than 7 pounds, less than 6 pounds, less than 5 pounds, and so on. In a particular embodiment, the volume of the device may be less than 1000 cm³, less than 900 cm³, less than 800 cm³, less than 700 cm³, less than 600 cm³, less than 500 cm³, and so on.

In an embodiment the device may include pre-programmed settings for the delivery of fluids such as nutritional fluids for feeding, including feeding times, frequency, speed, duration, and other settings, although the settings may be manually controlled in real-time by a user, caregiver, or medical provider. The programmed settings may be accessed, set, or changed by a user, caregiver, or medical provider, including in real-time and/or remotely. In a particular embodiment, a plurality of pre-programmed settings may be created for fluid delivery to a user. In a non-limiting example, 5 pre-programmed settings may be created to provide options for different types of feeding treatments as a standard configuration, however, this should in no way be considered limiting as other pre-programmed settings configurations may be created to provide customized fluid delivery programming.

In an embodiment, the device may track trends of feeding including volume delivered, calories delivered, duration of nutrition fluid delivery per feeding episode, time of feeding, number of daily, weekly, or monthly feeds. In an embodiment, a Machine Learning algorithm may analyze collected data from each user, or from groups of users having similar characteristics, to determine changes to the pre-established and pre-programmed feeding session settings or to determine when fluid delivery parameters have changed sufficiently to create an alert that is transmitted to a user, caregiver, or medical provider. The trend analysis may be used to determine adequacy of the prescribed feedings and/or treatment, to determine necessary changes to the feedings and/or treatment, and to set standards for the patient or to a patient group.

In an embodiment, the device may provide continuous infusion of chemotherapy agents directly to organ systems, such as low, continuous flow of fluid to the organ systems. The device may be placed externally in a wearable configuration with access to the affected organ through an installed port, or the device may be placed or implanted surgically for direct organ access, and may be combined with other fluid reservoirs and delivery devices. such as a subcutaneous catheter or its equivalent.

In an embodiment, the device may facilitate the process of peritoneal dialysis. The device, in aspects, may eliminate the need for an IV pole and gravity delivered peritoneal dialysis fluids through the ability to pump peritoneal dialysis fluids through the pumping mechanism configured within the device. Accordingly, the device may facilitate the development and use of wearable and other mobile peritoneal dialysis systems.

In an embodiment, the present system comprises a pumping mechanism, a fluid reservoir, a controller, and a wearable garment designed to house the aforementioned components of the system. System embodiments discussed herein are configured in hardware, software, and/or user interface components, such as a display screen, configured to receive input, instructions, and/or data, which may then be accepted, rejected, or manipulated by the user, caregiver, or practitioner to deliver formula at proper operating criteria, including standard criteria or specific criteria for the particular user. The system may be capable of communicating with a remote electronic device, such as, by way of non-limiting example, a smartphone, computer, server, or the cloud, so that information may be input or reviewed on or by the remote electronic device. Some embodiments may link the system to a smart device, computer, laptop, server, smart watch, or other electronic device associated with the user, caregiver, and/or medical provider to provide for control of device operating parameters such as flowrate, volume to be administered, duration of administration, and scheduling of future nutritional fluid delivery. The system is capable of being tailored for unidirectional delivery of enteral nutrition formula to any point of a patient's digestive tract.

Specifically, in embodiments, the system comprises an enteral nutrition pump, wherein the pump is capable of providing improved portability because the pump may operate with the administration reservoir horizontally arranged in relation to the pumping mechanism. In embodiments, the pump is capable of providing improved efficiencies because the pump may produce necessary flow rates for delivering nutrition in a smaller device footprint. Further, the immersion pumping system is capable of being used for the delivery of enteral nutrition formula to a patient through nasal, gastric, or jejunal access.

In an embodiment, Foundational Technical Specifications may be as follows:

TABLE 1

| | |
|---|---|
| Rate | Maximum: 1000 milliliters per hour |
| | Intended: 1.00-600 milliliters per hour |
| Resolution | <1.00 milliliters |
| Accuracy | +/−5%, or 0.5 ml/hr (whichever larger) |
| Capacity | High Output: 1500 milliliters (volume allowed |
| | per pump head) |
| | Intended: 1000 milliliters |
| Fluid Viscosity | High Output: 1000 cP |
| | Intended: 1-200 cP |
| Priming | Autonomous, self-priming |

*Battery Life: This is a low-end specification for the desired lifespan of a driver. Continuous users may at some points require about 60-150 ml/hr for up to 24 hours per day. Satisfying this particular need may be solved using a separately designed driver, or a replaceable battery configuration.

In a non-limiting example, one configuration of Pump Connection Features may be as follows:

TABLE 2

| | |
|---|---|
| Outlet Cxn | ENFit Luer Lock (ISO 80369-3) for example |
| (patient side) | See Tubing Diameter |
| | (Or custom at later development stages) |
| Outlet Cxn | Luer |
| (pump side) | Flange Fitting |
| | Push-fit |
| Inlet Cxn | Option 1: Integrated Disposable with Bag |
| (bag side) | Dip tube, Drain aid, Screw cap |
| | Option 2: Luer, Flange Fitting, Push Fit |
| Inlet Cxn | Luer |
| (pump side) | Flange Fitting |
| | Push-fit |
| Tubing | Inner Diameter: 3.5 mm |
| Diameter | Outer Diameter: 4 mm |

In an embodiment, the system may additionally comprise a second mechanism that operates to force liquid or formula into the inlet of the immersion pump to allow for multiple configurations or placements of the fluid reservoir and pump. The second mechanism may include an apparatus capable of rolling the fluid reservoir over itself as the contents are delivered and/or emptied. The second mechanism may also include a series of plates that sequentially compress the fluid reservoir to concentrate liquid at a front end leading to the pumping mechanism, or may include a series of plates on a track that progress during the feed overtop of the emptying fluid reservoir.

In an embodiment, the system may have the ability to link patient data to an electronic medical record (EMR) containing one or more data fields of patient data associated with a particular patient for increased transparency of patient and clinician communication. This data link may also assist with monitoring the patient's progress and feedings, changing the specific control information for each of the feedings, such as amount, times, and duration, treating the patient, and overall to ensure compliance with an established therapy for the client. Linking to the EMR may also provide for the ability to report adverse events during feeding from the patient to the clinician, for example, to better track patient quality of life (QoL). The ability to link to the EMR may also serve to replace the need for monthly, periodic, or frequent checkups required for patient nutrition care, or possibly allow for remote checkups rather than in-person checkups. The system and data connection to a patient EMR may be capable of decreasing or eliminating routine visits that may be performed through a digital environment with clinically relevant information collected by the system and related software and hardware.

In an embodiment, the system may deliver chemotherapeutic agents similar to a hepatic arterial infusion (HAI) pump. A HAI pump is designed to provide a continuous and constant rate of chemotherapy drugs to the liver, which allows higher doses and reduced exposure for normal cells. The system may be surgically implanted for direct access to the patient system and connected to a catheter or through an adapted G-button connection for delivery directly to the liver. The system may additionally be altered to sit outside of the body, rather than being surgically placed beneath the skin. In a non-limiting example, the device may allow for continuous, low flow rate delivery of therapeutic agents to specific organ systems over the period of days and weeks.

In an embodiment, the device may facilitate better outcomes during treatment of peritoneal dialysis. Peritoneal dialysis relies on the infusion of dialysis fluid into the abdomen with a suspended fluid supply and gravity driven flow. Through the employment of the proposed device, the process may be facilitated by way of a portable or wearable delivery system. The device may additionally provide more customization to the infusion flow rate that may maximize or optimize patient comfort, safety, and efficacy, as well as tracking, management, and control of infusion during treatment. In aspects, the device would allow for patients to perform their needed dialysis wherever they may be without the need, for example, to transport large, inconvenient, or cumbersome equipment. Further, the device may monitor, record, and transmit a data record of use including time and date, flow rates, volumes, and composition of the dialysate fluids.

In an embodiment the instant innovation may be used as part of a proprietary feeding delivery system with enhanced capabilities such as, by way of non-limiting example, improved visual indicators and monitoring. Such a system facilitates patient data collection, analysis, and instrumentation of the device in numerous non-limiting ways.

In an embodiment, the system herein described may permit volume and/or rate tracking and verification for empirical study and/or to confirm pump sensor readings for total system performance. The system permits occlusion detection within the access device, providing data separate from pump system occlusion detection and applicable for users that are non-pump feeders. In an embodiment the device may incorporate communication capabilities utilizing fiber optic transmission cables for rapid transmission of information, one or more optical sensors for recognizing sediment, biofilm, and residue buildup in the lumen of the device, and/or one or more sensors for attachment recognition. Such attachment recognition sensors may provide, among other non-limiting functions, automatic feed initiation upon connection, automatic feed cessation upon disconnection, and/or a pre-configured flow rate making possible initial rapid priming and then switching to a patient-specific low flow rate as customized for a particular user or group of users.

In an embodiment the system utilizes visual indicators and a related user interface incorporating both light indicators visible through an external bolster and color changing Stoma Liner materials. Such color change may be used to indicate the presence of inflammation or an infectious event, or the presence of Gastric Leakage. In an embodiment a light pipe and/or fiber optic design is relied upon to send light signals to a Personal Alert Safety System (PASS) device, and back.

In an embodiment, the instant innovation offers the convenience of instant pump priming. Due to the device pump's immersion and/or direct linear connection in a fluid feed, a user may prime the pump of the instant innovation while the feeding device is connected to a patient. This one-step process provides convenience and removes user error from a device requiring pump priming prior to device connection to a patient.

In an embodiment, device attachment between the Stoma Liner and the Patient is enhanced by use of Stoma Liners fitted in multiple sizes from 8 Fr-26 Fr, with adaptable configurations to make a tight seal. Each Stoma Liner has one or more low profile inner and outer bladders that may be inflated if the device fit needs further securement. Device attachment between the Stoma Liner and the Access Device, such as an Enteral Feeding Device, is enhanced by use of the bladders on the interior lumen of the Stoma Liner device that may be inflated and/or deflated to grip a variety of pinch points along the Access Device. In such embodiment, the Stoma Liner bladders have rigid tips that engage “female” attachment points on the stem of the Access Device. These female attachment points are characterized by slight rigid indentation to “accept” the tips located on the Stoma Liner bladders. The Access Device has a slightly flared bottom for extra attachment security.

In an alternative embodiment, a user may have a G-button connection point installed through the skin and the organ membrane, where the organ may be the stomach, upper intestine, lower intestine, or other bodily membrane. The G-button connection may provide a secure connection point for the enteral feeding system that does not depend upon the use of bladders inflated within the stomach or intestine for securing the connection point for the enteral feeding system.

In an embodiment, the instant innovation incorporates a super-hydrophobic inner coating to limit the build-up of formula, medication, particulate, or other extraneous material. Such coating reduces the frequency with which the device cavities need be flushed with water. The device incorporates outer coatings suitable to enhance cleanliness and long-life, such as, by way of non-limiting example, those with silver nitrate, chlorhexidine silver, and/or licensed BlueGuard technology.

In an embodiment, the instant innovation may be introduced to a patient by one or more procedures replacing typical surgical, endoscopic, or radiological procedures. Such one or more procedures benefit from the availability of a handheld surgical instrument that is preloaded with sutures or staples and used to secure a primary device (PASS) in-situ, adhering the stomach lining and abdominal wall in the process. Such instrument deploys the necessary suture and/or staple arrangement to secure the device in-situ with one trigger pull. Once the device is securely attached to the patient, the procedure may be concluded with necessary following steps such as, by way of non-limiting example, application of bandages or disinfectant or device operational testing.

In an embodiment, the instant innovation may be utilized to facilitate other cross-tissue or cross-membrane fluid transfer such as acting as, by way of non-limiting example, a Central Line Access Portal for long term access for infusions, enabling Dialysis through shunt placement, fistula formation, central line dialysis, and/or peritoneal dialysis, enabling Chemotherapy as a hyperport access; acting as a Hydrocephalus shunt, and permitting Colostomy applications, including ostomy implant, among many other possible applications.

In an embodiment, the instant innovation may be used in veterinary medical applications to a similar or greater extent than that to which it is used in human applications. Indeed, the instant innovation may be used in any medical or non-medical application requiring penetration and throughput of a flexible membrane or tissue.

In an embodiment, the instant innovation may have one or more “virtual” and/or “telehealth” applications, in which diagnostic and/or prescriptive and/or technical updates and/or communications are sent to and/or from the instant innovation by means of radio, Bluetooth, and/or Internet connectivity. By way of non-limiting example, such virtual or telehealth application may include an integrated component within the application that allows for patients to reach immediate assistance in the form of a “hotline.” Such “hotline” may be used by a patient or caregiver in the event of an accident incurred during use of the instant innovation, or in the event that a patient or caretaker requires use instructions. In a non-limiting example, activating such hotline may include the patient's or the caregiver's clicking of a physical or virtual button and/or the patient's or the caregiver's submitting of a form with an incident report or request.

In an embodiment in which the service provider of the instant innovation has an internet website and/or a mobile app, one of the features of the website may allow a patient/caregiver to sign up for synchronous virtual sessions with a care team. Such synchronous virtual sessions would integrate into the patient's care regimen the care team's scheduling and video application. In an embodiment, the instant innovation may offer an asynchronous option in which a patient and/or caregiver can upload a video onto the service provider's website describing a particular feeding experience, whether it be positive or negative.

In an embodiment the instant innovation may permit remote patient monitoring of the in-line or other pump flow rate, along with feedback-based flow rate improvement. Feedback useful in flow rate improvement may include patient notes as to whether a particular feed was satisfactory, tolerable or unacceptable. Such normative judgment may be based in part on patient-reported feelings of fullness, bloat, sickness, unease and/or other qualitative effects of a feed.

In an embodiment the instant innovation may include one or more patient or caregiver training components. By way of non-limiting example, the instant innovation may include a virtual training assessment on the device, such virtual training assessment requiring the user to correctly assemble the device and to pump a “dummy” packet of water or solution to verify the user's understanding of the device functionality. Such an assessment may provide prompts to “walk” a patient or caregiver through each step of the process. Each step of the process may be illustrated with virtual diagrams and/or videos. In the presence of an assessment-enabled embodiment, the device may have stage-gated features, such that only those users that have successfully passed device training may access full device functionality.

The instant innovation may offer unique benefits to hospitalized or home-bound patients. In an embodiment, Luminoah software may include a simulated use feature on the mobile application for patients or caregivers to prepare for the patient's transition to home. Such software may include a simulation setting, that would allow the patient or caregiver to set a proposed feeding regimen and delineate any necessary requirements. In the weeks and/or days leading up to a hospital patient's discharge to home, the app of the instant innovation may send reminders and/or “calls-to-action” to the patient and/or caregiver to have the patient and/or caregiver go through the process of setting up and starting a feed. Such latter described process may initiate and/or reinforce good feed administration practices using tracking/reminder features, and/or features of the app that may require manual user input.

In an embodiment, the instant innovation may incorporate a hospital-based training application. Such application may operate similarly to the above-described process, except for all device features being fully controlled by the hospital nursing staff. Such application would allow patients and/or caregivers to have a fully nurse-controlled “trial-run” of operations prior to the patient's discharge from the hospital. Operation of the application by a nurse for a few days prior to discharge may suffice to help identify and offer solutions for difficult or unexpected feed issues.

In an embodiment using a nutrition pack and a G-button device, the instant innovation may check, during any application-directed simulation, if the device tubing set itself has been placed properly onto the device drive shaft and if the device tubing set is properly connected to the nutrition pack and the G-button. In this and other embodiments, caregivers benefitting from any and all training may include school nurses or other personnel who manage care for enteral nutrition-receiving patients.

In an embodiment, the instant innovation may include one or more predictive or data analytic features. Such predictive or data analytic features may incorporate one or more machine learning algorithms to predict a unique normal and tolerable feeding rate for individual patients to use.

In an embodiment, the instant innovation may incorporate a predictive or data analytic feature directed to predicting which feed type would be the best for a particular patient based on that patient's particular medical history. In such embodiment, a predictive or data analytic algorithm may receive as input numerous and various data features from a patient's medical history to determine which feed type would be best suited to the patient based upon factors including but not limited to time of day and/or time of year, patient's weight, mood, or physicality, or patient's nutritional or emotional needs, including receptiveness to feeding.

In an embodiment, the instant innovation may include a predictive or data analytic algorithm to set a patient's feeding schedule based on the various flow rates and volume of nutrition the patient is given. Such calculated feeding schedule may be pre-programmed into the feed controller, with the patient being given a number of options as to how he/she wants to split the feeds or determine the duration of each feed. By way of non-limiting example, if a patient had a tolerable flow rate of 100 mL/hr. and a required 500 mL of nutrition every day, then the algorithm may calculate regimen options such as the patient's having two feeds twice a day with 250 mL per feed for 100 mL/hr. or 4 times a day with 125 mL per feed at 100 mL/hr. In this or other embodiments, the instant innovation may include a simple counting function where the patient and/or caregiver may input the amount of feeding supplies on-hand to determine in part the sufficiency of such supplies for feedings administered over any given period. By way of non-limiting example, such function may determine that 100 200 mL nutrition packs would last X number of days based upon the particular patient's unique feeding regimen.

In an embodiment, the instant innovation may include a predictive or data analytic algorithm to analyze the data from a patient's feed rate to determine the presence of evidence suggesting bloating, regurgitation, and/or other side effects from a feed. A model resulting from such analysis may predict if a particular patient will experience bloating or other side effects during any subsequent feeding and may permit prospective as well as reactive feed adjustment to minimize unwanted feed outcomes. The predictive model may continuously learn with input of newly-generated patient data.

In an embodiment, the instant innovation may be integrated with one or more other Smart Home Health devices in order to create trends from data derived outside the feed system that would not be possible with feeding data alone. Non-limiting examples of such devices may include BMI tracking with Smart Scales; Heart rate/activity monitoring with a Smart Heart Monitor; aspiration or regurgitation event monitoring with a Smart Pulse Oximeter; and blood sugar monitoring with a Smart Glucose Monitor. In this and other embodiments, the instant innovation may include a Gyroscope and/or Accelerometer for physical activity monitoring that would permit automatic adjustment of feeding rate based upon kinesthetic indications. By way of non-limiting example, the instant innovation may reduce the rate of feeding when such gyroscopic and/or accelerometric data suggest that the patient is lying down as opposed to sitting upright, or as opposed to standing upright and moving.

In an embodiment, the instant innovation may incorporate one or more alarms and/or alerts to prompt patient and/or caregiver action. These alarms and/or alerts may be communicated any number of ways including, by way of non-limiting example, visually, aurally, and/or tactilely. Tactile communication may include haptic feedback from the innovation device as a primary or alternate alarm. By way of non-limiting examples, such haptic feedback may be initiated when the device pump is accurately placed onto the drive shaft, and/or when the device pump is accurately assembled to include all device disposables.

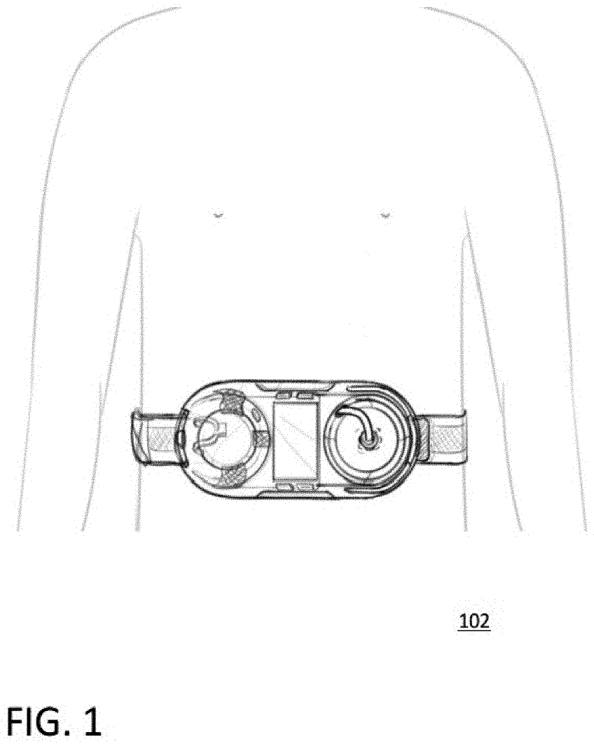

Turning now to FIG. 1, a view of the device in-situ upon a patient's torso consistent with certain embodiments of the present invention is shown. At 102 is a belt-mounted nutrient reservoir, electronic control panel, motor, pump, and cross-cutaneous access point assembly removably attached across a patient's midsection.

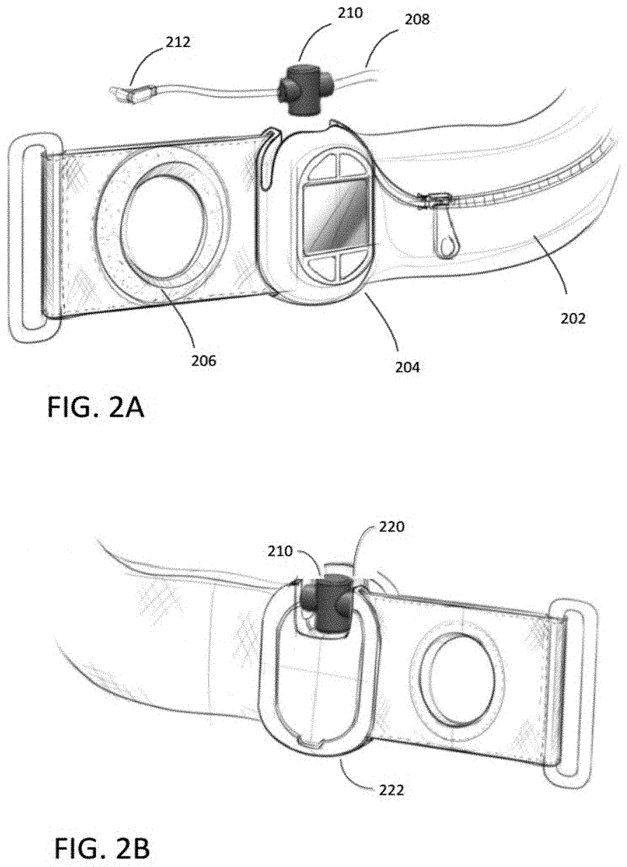

Turning now to FIG. 2A, a partially exploded front view of the device consistent with certain embodiments of the present invention is shown. At 202 is a nutrient reservoir which connects to disposable pump head 210 by operation of Inlet Connection 208. Flow of enteral nutrition from a nutrient fluid reservoir 202 is affected and metered by electrical control 204. Electrical control 204 may include a reusable pump motor (not shown). Outlet Connection 212 connects the patient-proximal end of pump head 210 through ring 206 to patient cross-cutaneous access point (not shown).

Turning now to FIG. 2B, a rear view of the device consistent with certain embodiments of the present invention is shown. Here, the assembly of FIG. 2A is shown from the side of the assembly intended to be worn against a user's body. The disposable pump head 210 is seated within a pump housing 220, which may be configured for insertion of the pump head 210 to securely hold the pump head 210 in place during fluid transfer operations. The electrical and control housing 222 may be placed in contact with the wearer when the system is worn by the user.

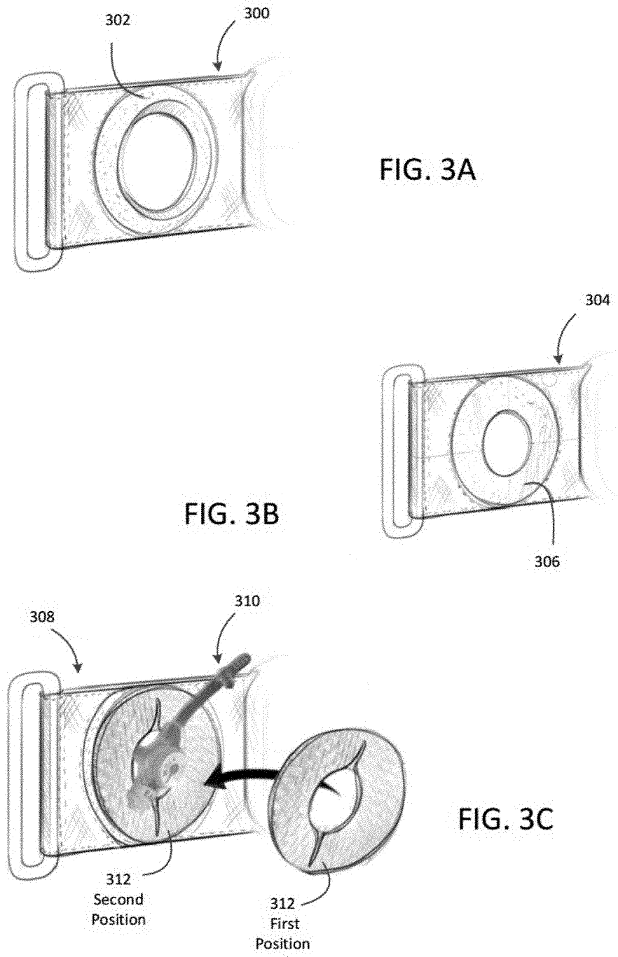

Turning now to FIG. 3A, a first detail view of the cross-cutaneous access portion of the device consistent with certain embodiments of the present invention is shown. At 300 is the belt assembly to which hard elevated ring 302 is attached. Elevated ring 302 may be made of a wide variety of suitable materials including but not limited to nylon, plastic, and/or metal. Choice of material for elevated ring 302 may be based in whole or in part upon strength, use, and cleanliness considerations.

Turning now to FIG. 3B, a second detail view of the cross-cutaneous access portion of the device consistent with certain embodiments of the present invention is shown. At 304 is the belt assembly to which soft ring 306 is attached. Soft ring 306 may be made of a wide variety of suitable materials including but not limited to fabric, cloth, and/or foam. Choice of material for soft ring 306 may be based in whole or in part upon strength, use, and cleanliness considerations.

Turning now to FIG. 3C, a third detail view of the cross-cutaneous access portion of the device consistent with certain embodiments of the present invention is shown. At 308 is the belt assembly through which is threaded pump head assembly 310. At 312 is a loop-and-hook-fastenable patch capable of being removably positioned from a first position not in contact with belt assembly 308 to a second position affirmatively in contact with belt assembly 308.

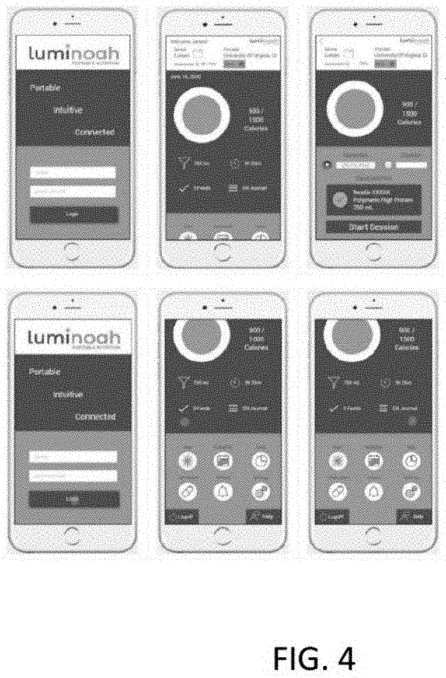

Turning now to FIG. 4, several smart-device-integrated user experiences consistent with certain embodiments of the present invention are shown.

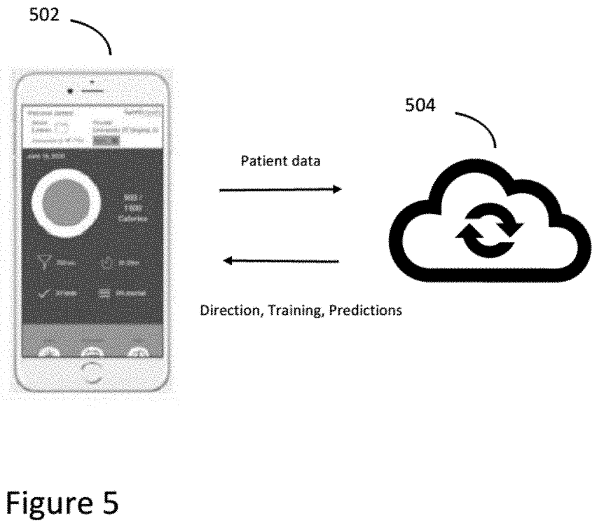

Turning now to FIG. 5, patient-remote diagnostic and therapeutic communications consistent with certain embodiments of the present invention are illustrated. Smart device 502 sends patient data to one or more servers 504. Patient data may include, by way of non-limiting example, patient feed rate, self-reported patient experience data, and patient feed type, time, and frequency. One or more servers 504 perform data analytic functions in support of virtual telehealth communications, patient and/or caregiver training, and patient-need predictive applications. One or more servers 504 send direction, training, and/or predictions to smart device 502 for therapeutic implementation.

While certain illustrative embodiments have been described, it is evident that many alternatives, modifications, permutations and variations will become apparent to those skilled in the art in light of the foregoing description.

We claim:

1. A liquid-delivery apparatus comprising:
an apparatus housing a pump mechanism, at least one liquid reservoir, connection tubing, a disconnectable connector, said disconnectable connector configured to be inserted into a cross-cutaneous access point;
said liquid-delivery apparatus further capable of housing communication and control electronics;
one or more sensors for detecting and recording liquid delivery operation parameters;
said liquid-delivery apparatus capable of sustainably supplying, tracking, and managing a flow of liquid through said pump mechanism to said cross-cutaneous access point; and
where said liquid-delivery apparatus creates regimen-predictive analytics and one or more recommendations for user adjusting operation of the apparatus in response to said regimen-predictive analytics to customize apparatus operation for said user.

2. The apparatus of claim 1, where said at least one liquid reservoir comprises a pouch of liquid for treating a user physical condition.

3. The apparatus of claim 2, where the user physical condition requires enteral nutrition, medication, or delivery of other beneficial liquids.

4. The apparatus of claim 1, where the communication and control electronics further comprises network connectivity, control and management software modules, and a display presenting information that is visible to a user, medical professional, colleague, or family member.

5. The apparatus of claim 1, where said pump mechanism is at least one of an inline or immersion pump head that is disposable.

6. The apparatus of claim 5, where said disposable pump head is operated by an electric motor or mechanical driver.

7. The apparatus of claim 1, where said sensors are connected to the flow of liquid through the pump mechanism, connection tubing, and cross-cutaneous access point to collect measurement and operational data.

8. The apparatus of claim 1, further comprising a semi-permeable membrane into which a cross-cutaneous connector is inserted to establish a lockable connection.

9. The apparatus of claim 1, where said pump mechanism is operable to pull liquid from said liquid reservoir, pass said liquid through said connection tubing, and push said liquid through the cross-cutaneous access point for delivery to a user.

10. The apparatus of claim 1, where said connection tubing further comprises tubing connecting said liquid reservoir to said pump mechanism, and tubing connecting said pump mechanism to a cross-cutaneous connector.

11. The apparatus of claim 1, where said communication and control electronics further comprise an electronic storage repository into which all collected sensor data is stored.

12. The apparatus of claim 11, where said communication and control electronics connect through a network connection to transmit said collected sensor data to an outside data processor.

13. The apparatus of claim 1, where said liquid-delivery apparatus is a wearable apparatus and further comprises a loop-and-hook-fastenable patch capable of being removably positioned from a first position not in contact with said wearable apparatus to a second position affirmatively in contact with said wearable apparatus.

14. The apparatus of claim 13, where said wearable apparatus can be any of a belt, band, pouch, or other apparatus capable of encircling a portion of the user's body.
